(19) United States
(12) Patent Application Publication
(10) Pub. No.: US 2015/0106024 A1
(43) Pub. Date: Apr. 16, 2015

(54) SYSTEMS AND METHODS FOR DETERMINING IMPLANT POSITION AND ORIENTATION

(71) Applicant: Orthonetic, LLC, Dover (DE)

(72) Inventors: Christopher Lightcap, Davie, FL (US); Jonathan Azevedo, Fort Lauderdale, FL (US)

(21) Appl. No.: 14/511,612

(22) Filed: Oct. 10, 2014

Related U.S. Application Data
(60) Provisional application No. 61/889,272, filed on Oct. 10, 2013.

(51) Int. Cl. A61B 5/00; G01B 21/16; A61B 5/11; A61F 2/46

(52) U.S. Cl. CPC A61B 5/4851; A61F 2/468; A61F 2/4657; G01B 21/16; A61B 5/11
USPC 702/19; 702/150; 600/587

(57) ABSTRACT

A method for determining implant position and orientation comprises generating a plurality of predetermined criteria associated with a surgical procedure. The plurality of predetermined criteria including at least one of a mechanical alignment metric, a soft-tissue balancing metric, and a functional outcome metric. The method also comprises receiving one or more user selections of performance criteria, the one or more user-selections based on a user's desired outcome of the surgical procedure. At least one weighting factor associated with a simulation algorithm may be adjusted based on the received user selections of predetermined criteria. The method also includes simulating a patient-specific model, and determining performance metrics based on the user selected performance criteria. The information indicative of at least one of a recommended implant position or a recommended implant orientation may be provided for display to a graphical user interface, the information being based on the performance metrics.

### CROSS-REFERENCE TO RELATED APPLICATION

This application claims the benefit of U.S. Provisional Application No. 61/889,272, filed Oct. 10, 2013, which is incorporated herein by reference in its entirety.

### TECHNICAL FIELD

The present disclosure relates generally to orthopedic surgery and, more particularly, to systems and methods for determining implant position and orientation based on simulations using patient-specific data.

### BACKGROUND

Recent advances in computer-assisted surgery have enabled surgeons to precisely execute a pre-operative plan to unprecedented levels of accuracy. Such techniques may utilize computer-assisted navigation systems, e.g. optical tracking, electromagnetic tracking, and/or precise robotic manipulators to help the surgeon execute his plan. However, despite this newly-achieved level of accuracy, many of these systems do not sufficiently account for patient-specific dynamics in the surgical planning, relying instead on generic characteristics of joint kinematics.

For example, the two most common surgical techniques for knee arthroplasty are referred to as measured resection and gap balancing. Both techniques replace diseased or damaged joint surfaces with metallic and plastic components in order to relieve pain and restore motion.

In a gap balancing technique, the surgeon may release ligaments or adjust the implant position to result in balanced medial and lateral tibiofemoral distances or “gaps”, in both flexion and extension. The surgeon may correct laxity in one or more compartments and/or imbalance by adjusting the position and/or orientation of the implant, at the expense of raising or rotating the joint line. The gap balancing approach may consider the surrounding soft tissues by measuring tibiofemoral contact forces (e.g. using a strain-gage instrumented trial) or joint displacements (e.g. using conventional navigation techniques), but this approach does not compute an optimal implant position and orientation to balance medial and lateral gaps throughout range of motion, nor does it consider the patient-specific kinematics or dynamics.

In a measured resection technique, the surgeon makes precision cuts to the bone and aligns the implant based on the bony anatomy, e.g. the anatomical axis, transepicondylar axis, and/or posterior condylar axis. During reconstruction of the joint, the surgeon aims to replace the exact thickness of the resected portions to ensure that the reconstructed anatomy (and the reconstructed axes) matches the original anatomy of the joint as closely as possible. The theory behind measured resection is that, because everything that is removed is replaced, the original (and ideal) knee balance is restored. One benefit of this technique is that the femur and tibia can be resected independently of one another, so long as the position of the reconstructed axis is maintained.

The conventional techniques for implant planning are not sufficiently equipped to consistently restore alignment, joint balance, and function as desired by the surgeon. Indeed, they either assume bone kinematics remain unchanged for varying implant positions or consider only static geometric constraints, e.g. reference to bony anatomy, and not any dynamic loading conditions.

The present disclosure, hereafter referred to as patent-specific implant planning, provides a system for determining an optimized implant position and orientation given a set of performance criteria and constraints provided by the surgeon and a patient-specific computational model. This technique provides a solution to the problem of determining the correct implant pose, and compliments the existing computer-assisted and robot-assisted surgical techniques which already possess the capability to accurately reproduce a surgical plan. Regardless of whether the surgeon seeks to restore constitutional alignment or adjust the joint to neutral mechanical alignment, the patient-specific implant planning technique informs the ideal alignment to balance the soft-tissue structures during static and dynamic conditions.

The present disclosure solves one or more of the problems in conventional implant planning systems by disclosing a technique for calibrating a patient-specific computational model either pre- or intra-operatively, performing dynamic simulations given a patient-specific computational model and implant position, and determining an implant pose or resection plan based upon surgeon-defined metrics and constraints.

### SUMMARY

According to one aspect, the present disclosure is directed to a method for determining implant position and orientation. The method may comprise recording at least one kinematic or kinetic parameter during a passive loading of a portion of an anatomy associated with the joint of a patient. The method may also comprise calibrating a patient-specific software model associated with a patient's joint based, at least in part, on the at least one kinematic or kinetic parameter, and receiving, by the processor, one or more user-selected parameters associated with joint performance. The method may further comprise simulating performance of the patient's joint using the calibrated patient-specific model and the one or more user-selected parameters. The method may also comprise providing, by the processor, information indicative of at least one of a recommended implant position or a recommended implant orientation, based on the simulated performance.

According to another aspect, the present disclosure is directed to a method for devising a resection plan for reducing joint impingement, comprising calibrating a patient-specific software model associated with a patient's joint based, at least in part, on one or more of: a geometry of the patient's joint, a kinematic parameter of the patient's joint, or an external reaction forces associated with the patient's joint. The method may also comprise receiving, by the processor, one or more user-selected parameters associated with joint performance and simulating performance of the patient's joint using the calibrated patient-specific model and the one or more user-selected parameters. The method may further comprise generating information indicative of a resection plan associated with the patient's joint, based on the simulated performance.

According to yet another aspect, the present disclosure is directed to an apparatus for measuring external reaction forces used in calibrating a patient-specific model. The apparatus may comprise a leg holding device configured to receive at least a portion of a patient's lower leg. The apparatus may also comprise a plurality of sensors coupled to the leg holding device and configured to measure an external force applied to the patient's lower leg. The apparatus may further comprise a tracking device coupled to the leg holder and configured to locate at least one of a position or an orientation of the leg holding device relative to an anatomical feature of the patient.

### BRIEF DESCRIPTION OF THE DRAWINGS

FIG. 1 provides a diagrammatic view of an exemplary computer-assisted surgical environment, consistent with certain disclosed embodiments;

FIG. 2 provides a diagrammatic perspective view of a device for measuring the external reaction forces presented to the patient during a calibration phase, in accordance with certain disclosed embodiments;

FIG. 3 provides a flowchart illustrating an exemplary process for implant pose optimization, consistent with certain disclosed embodiments;

FIG. 4 provides a flowchart illustrating another exemplary process for implant pose optimization and selection, in accordance with certain disclosed embodiments;

FIG. 5 provides an exemplary graphical user interface for allowing selection/customization of certain implant pose positioning criteria, which may be based on patient-specific criteria, consistent with certain disclosed embodiments;

FIG. 6 provides an exemplary graphical user interface for allowing selection/customization of certain implant pose positioning criteria after simulation, in accordance with certain disclosed embodiments;

FIG. 7 provides a flowchart illustrating an exemplary process for calibrating patient-specific models and corresponding output models associated therewith, consistent with certain disclosed embodiments;

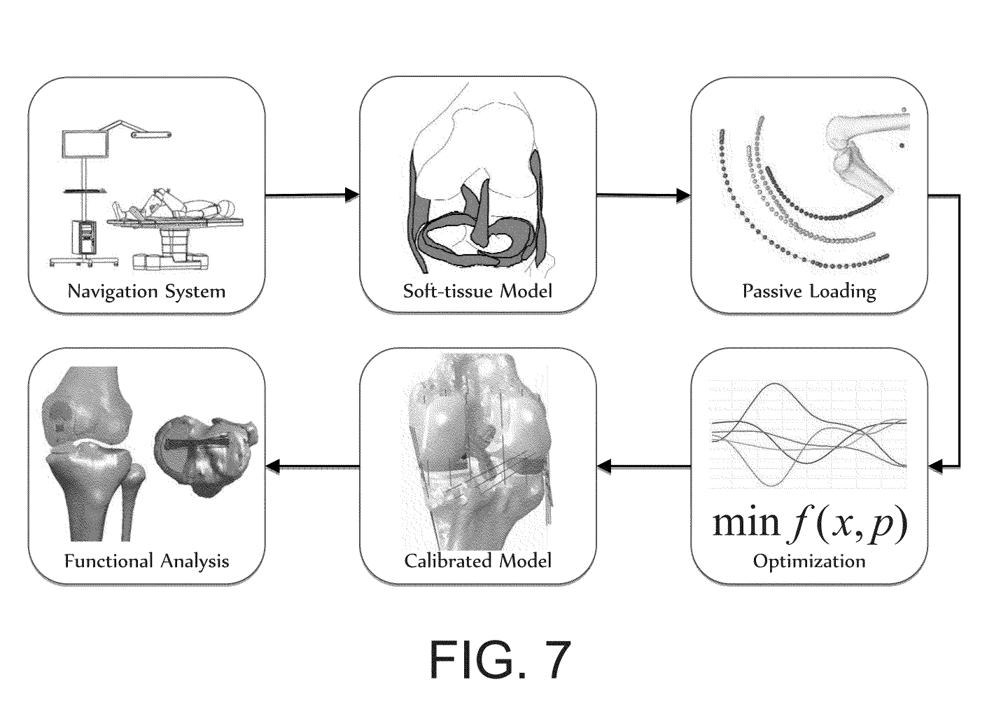

FIG. 8 illustrates exemplary subcomponents and non-limiting description of the corresponding subcomponents, in accordance with certain disclosed embodiments;

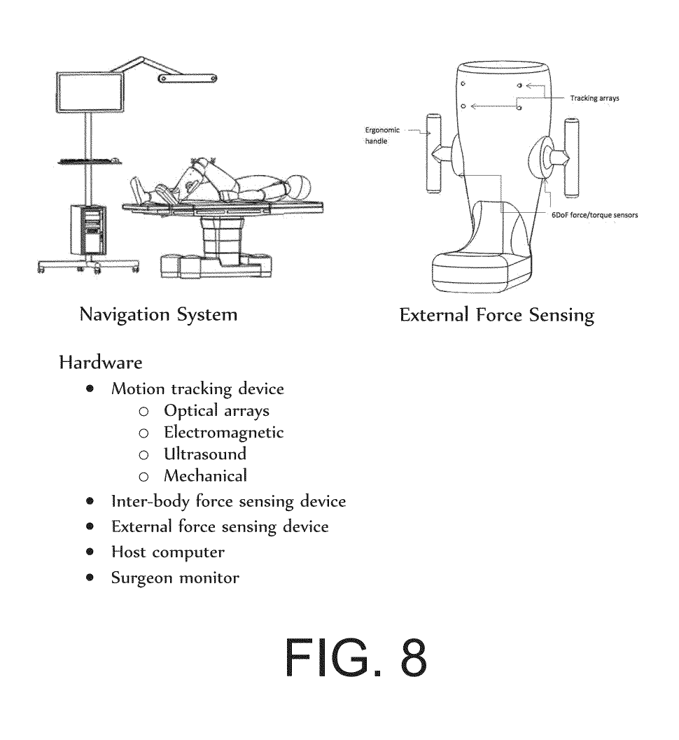

FIG. 9 depicts certain soft-tissues of a knee joint and outlines exemplary data that is used to formulate a soft-tissue knee model that can be used in accordance with the disclosed embodiments;

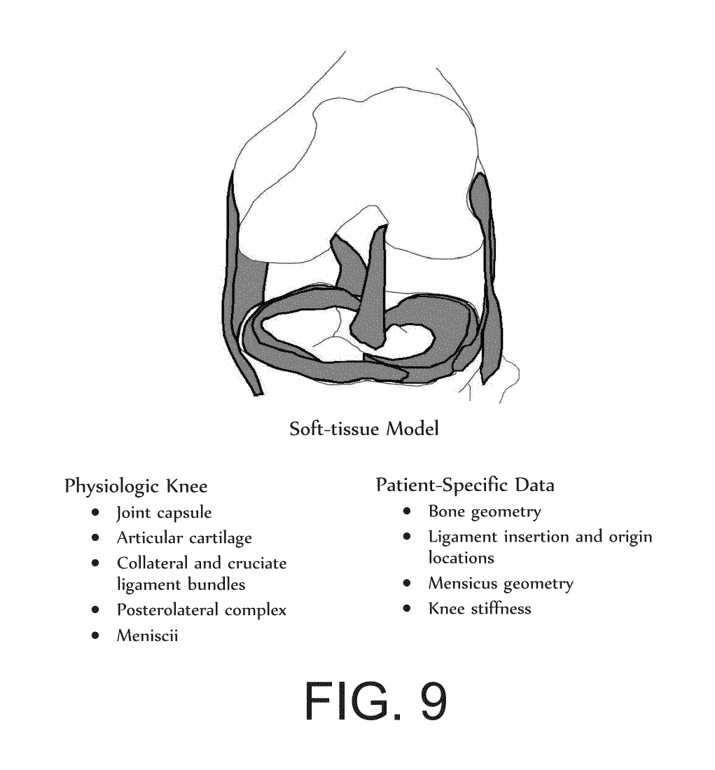

FIG. 10 outlines exemplary data that can be used to compute the parameters of the patent-specific knee model, consistent with certain disclosed embodiments;

FIG. 11 provides an illustration depicting the optimization and tuning framework for determining patient-specific parameters, which can be implemented consistent with the disclosed embodiments;

FIG. 12 outlines exemplary features associated with a patent-specific model created and optimized consistent with the disclosed embodiments;

FIG. 13 outlines exemplary features and uses of the patent-specific model generated in accordance with the disclosed embodiments; and

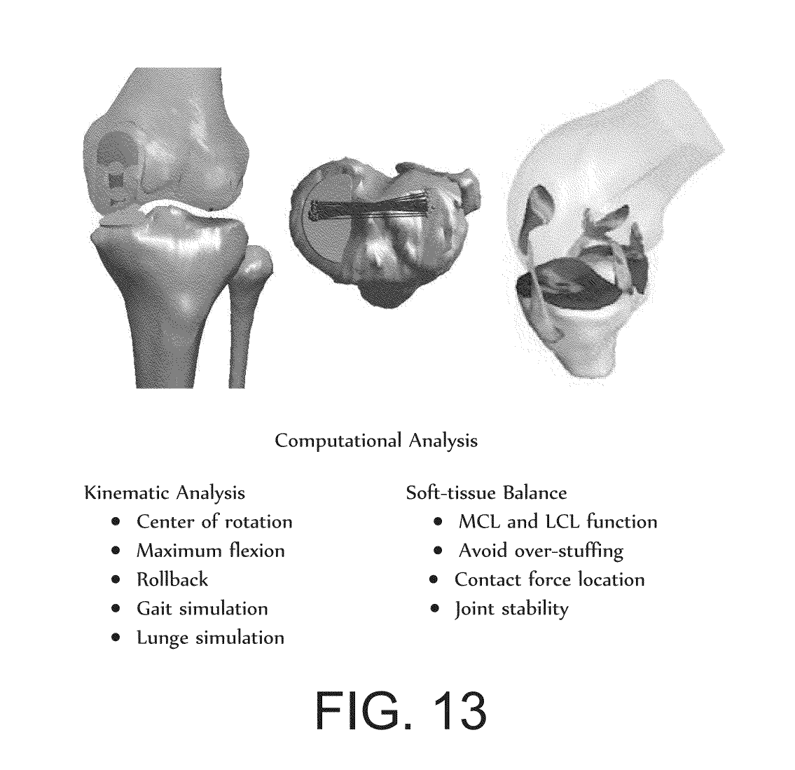

FIG. 14 illustrates an exemplary processor-based computer system, on which certain methods and processes consistent with the disclosed systems and methods for determining implant position and orientation may be implemented.

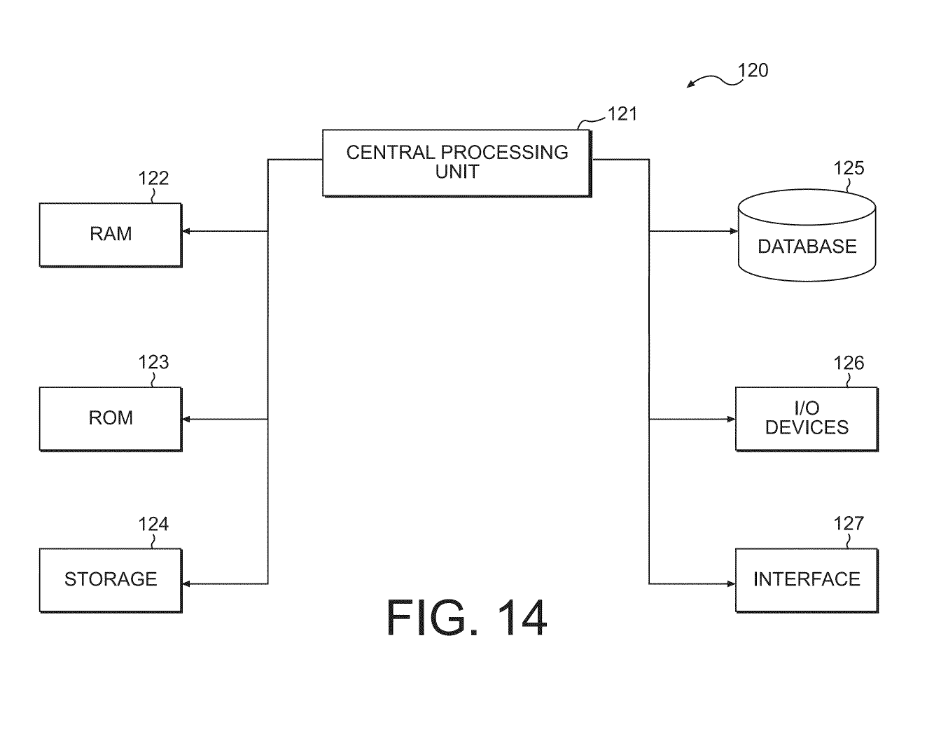

### DETAILED DESCRIPTION

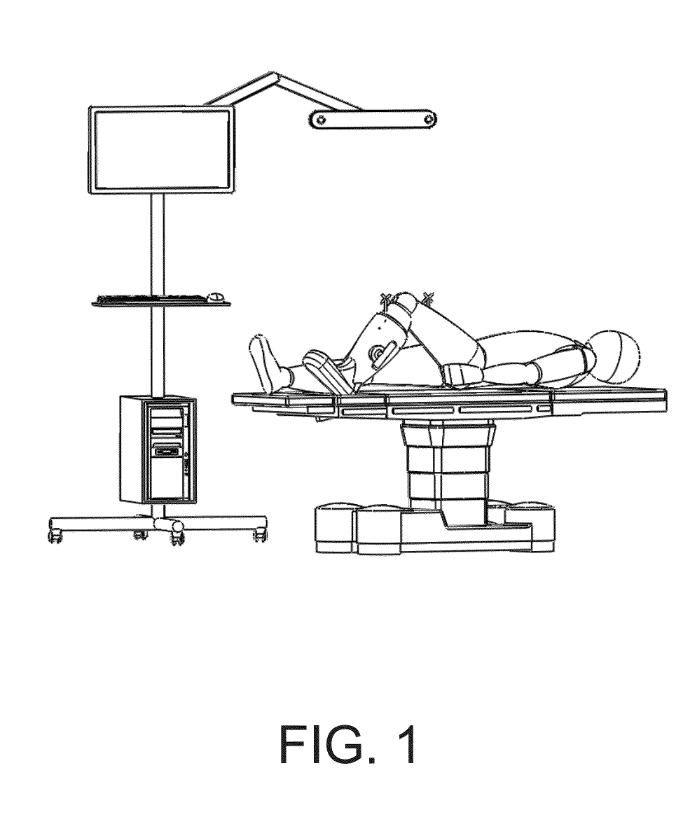

FIG. 1 provides a diagrammatic illustration of an exemplary computer-assisted surgical environment, in which the presently disclosed systems and methods for determining implant position and orientation may be implemented. According to one embodiment, and as illustrated in FIG. 1, the surgical environment may comprise the following hardware: a motion tracking device, an inter-body force sensing device, an external force sensing device, a host computer, and a surgeon monitor. Although not necessarily illustrated in FIG. 1, those skilled in the art will appreciate that the surgical environment may also include software programming and computing capabilities required to execute the intra-operative knee calibration and implant pose optimization, such as those that will be described below and any other software (e.g., calibration software, etc.) that may be incident to the proper performance of a particular surgical system.

According to one embodiment, motion tracking devices, inter-body force sensing devices, and external force sensing devices may each be communicatively coupled to a host computer, either wired or wirelessly. Each of these systems may also be configured to provide real-time measurements of joint kinematics and dynamics. A surgeon monitor may be connected directly to the host computer to display a 3D rendered image of the patient anatomy and implant, along with real-time patient kinematics and dynamics, patient-specific model parameters, and optimized implant pose. In an alternative embodiment, a tablet computer, such as an iPad or tablet PC, may be connected via a wired or wireless connection to the host computer, and display such information to the surgeon.

The present disclosure may also include a database or other storage device that stores therein pre-operative patient-specific data; a motion tracking and/or force sensing instrumentation to monitor patient kinematics, inter-body forces, and/or external reaction forces; a computing device to determine an implant pose, and a computer display to presents the results.

In an exemplary embodiment, pre-operative patient-specific data may include a pre-operative CT scan of a patient's anatomy. A 3D model of the anatomy may be generated through conventional segmentation and reconstruction methods. In an alternative embodiment, an MRI scan may be performed to identify the ligament origin and insertion sites on the bone, which may later be used in the computational model.

In one embodiment, the motion tracking device may be an optical tracking system, such as one commercially distributed by Northern Digital Inc. (NDI), and configured to provide real-time measurements of tracking arrays through a USB interface. Optical tracking systems, comprising optical cameras and optical tracking arrays, may be designed for passive retro-reflective arrays or active LED arrays. Optical tracking arrays may be rigidly attached to the bone using one or more surgical bone pins, in order to accurately monitor patient-specific kinematics in real-time. An apparatus may be designed to position the camera in an optimal position and orientation, as to minimize occlusions from the surgeon and his/her assistants. Furthermore, said apparatus may include an enclosure to locate a computer for reading patient-specific kinematic and dynamic measurements, and a monitor for displaying such information to the surgeon. Any number of computer interface devices, such as a mouse, keyboard, or camera, may be used to interact with this system. Other examples of motion tracking devices include electromagnetic, ultrasound, and mechanical tracking devices, e.g. passive articulated arm coordinate measuring machines (AACMM).

In one embodiment, the computer may also include a wireless card for reading from one or more wireless devices, such as an inter-body force sensing device, and external force sensing device. In an alternative embodiment, the computer may be wireless connected to a tablet computer, such as an iPad or tablet PC, as the primary point of interaction.

The computer may be configured to run an operating system, such as Windows, Linux, or Mac OSX, having USB and network device drivers to interface to the hardware. A computer program may be developed using any number of standard IDE tools, such as Visual Studio or Xcode, and may be configured to provide real-time 3D image rendering through software libraries such as OpenGL or Direct3D. Higher-level OpenGL frameworks may also be incorporated to reduce development time, e.g. GLUT, VTK/ITK, or Java3D. A computer program may include calibration and optimization algorithms utilizing commercially-licensed or open-source numerical integration, optimization, or finite-element modeling software libraries.

The computer-assisted surgical environment may also include a device for measuring the inter-body forces of the knee. For example, such a device may utilize ultrasonic piezoelectric sensors to compute the magnitude and center of pressure of inter-body forces in the knee joint, e.g. the Verasense™ knee balancer (OrthoSensor Inc.). In an alternative embodiment, the device may utilize paper-thin pressure sensors to compute the magnitude and center of pressure of the medial and lateral contact forces, e.g. the K-Scan™ joint analysis system (Tekscan Inc.). In yet another embodiment, the device may utilize strain gauge measurements to compute the magnitude of the medial and lateral contact forces, e.g. the eLibra Dynamic Knee Balancing System™ (Synvasive Technology, Inc.).

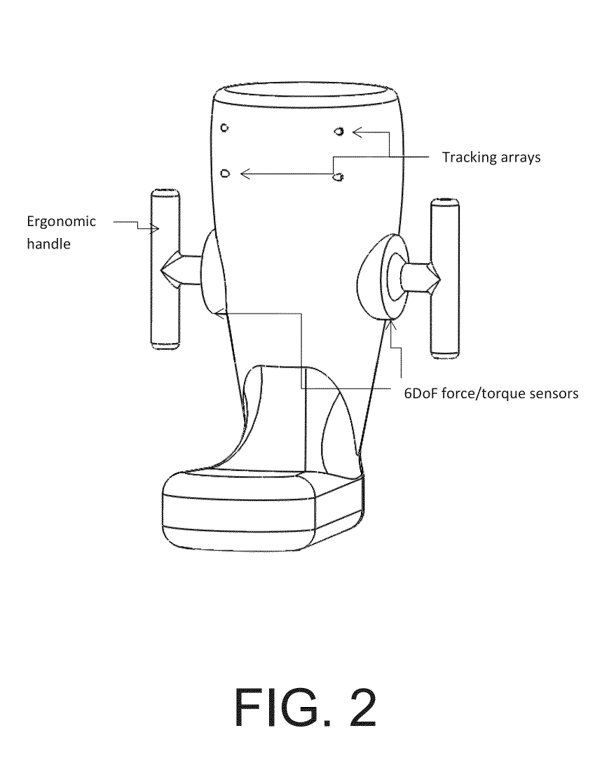

In addition to intra-body forces, the computer-assisted surgical system or environment may include a device for measuring the external reaction forces presented to the patient during a calibration phase. As illustrated in the exemplary embodiment of FIG. 1, this device may embody a rigid boot attached to the patient's foot. As shown in FIG. 2, the device may have two handles for manipulating the patient's leg, where a collection of strain gauges may be mounted on a beam which connects the handles to the boot in order to estimate the strain, and therefore 6 degree-of-freedom (DoF) forces and torques, exerted by the surgeon throughout a passive range of motion. The device may also include a tracking device, such as an optical tracking array, in order to locate its position with respect to the patient's leg. In an alternative embodiment or in addition to the boot shown in FIG. 2, the device may include a traditional leg holder which has been modified to include sensors for measuring the external forces applied to the patient's anatomy during surgery. The leg holder may also be an actively-controlled robotic manipulator, for which the external reaction forces may be computed directly from motor currents or joint torque sensors.

In an alternative embodiment, a rigid horseshoe-shaped collar may be placed underneath the thigh to measure external reaction forces and provide another interaction point for the surgeon. The rigid collar may also be instrumented with a collection of strain gauges to estimate the resulting 6-DoF forces and torques exerted by the surgeon with respect to a local coordinate system. In addition, a tracking array, such as an optical, EM, or ultrasound array may be used to locate the collar, and its measured forces, with respect to the patient's leg.

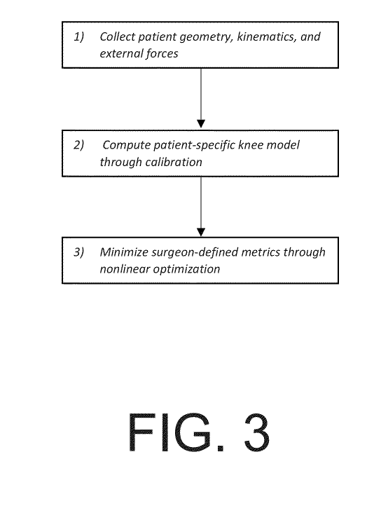

The present disclosure may also include a host computer for collecting and managing data from the constituent devices and subsystems, and to compute a patient-specific implant plan. The host computer may be configured to perform a method to determine an implant pose. As illustrated in FIG. 3, such a method may comprise three basic processes: collecting relevant patient geometry, kinematics, and forces; determining a patient-specific model (e.g., through calibration); and minimizing a surgeon-defined metric through optimization to achieve a desired implant plan.

The presently-disclosed process for determining an optimal patient-specific implant position and orientation generally comprises a number of steps. First, a patient-specific computational model, or knee model, must be computed pre-operatively or intra-operatively to simulate the behavior of the human knee. In one embodiment, the knee model exists as a mathematical formulation, algorithm, or numerical process residing in computer software. The primary objective and inherent function of the knee model is to predict patient-specific knee kinematics, kinetics, and relative soft-tissue behavior. According to one embodiment, the knee model may be calibrated, using a software program, to pre- or intra-operatively collected passive knee response data in order to determine the patient-specific knee model parameters (e.g. ligament origin and insertion sites) that may otherwise be difficult to obtain and measure without causing irreversible damage to the patient. The knee model and its parameters are subsequently used in the present invention to assist the surgeon in developing an optimized plan for knee arthroplasty.

According to an exemplary embodiment, the knee model may comprise three components: a set of input parameters, a set of output parameters, and a system of equations that mathematically relate the input and output parameters. Input parameters may include but are not limited to bone geometry data, knee joint kinematics, knee joint kinetics, and knee joint biomechanical material properties.

Bone geometry data may be obtained from segmentation and reconstruction of computed-tomography (CT) and/or magnetic resonance medical imaging prior to surgery. The geometrical data representing the bony surfaces may be stored as polygonal meshes (e.g. discrete sets of three-dimensional vertices and surface normal vectors). Alternative, analytical spline functions may be fit to such surface points to form more compact and continuous representations.

Knee joint kinematics may be measured using conventional computer-assisted surgical techniques. For example, in one embodiment, optical motion tracking systems and bone trackers may be utilized to accurately track the position and orientation of the patient's bony anatomy in real-time. A registration procedure is commonly incorporated by such systems to relate the position and orientation of the bone trackers to the reconstructed bone geometry.

Knee joint kinetics may be obtained via an external force sensing device in contact with the patient. This device may be a rigid brace or boot with handles for grasping and providing measurable forces and moments to the knee joint. Alternatively or additionally, an inter-body force sensing device (e.g. a VeraSense or eLibra device), may also provide such kinetic information to the knee model.

Knee joint biomechanical material properties may be obtained from published mechanical testing literature. These properties define patient-specific material models for modeling ligaments, articular cartilage, meniscus, and capsular structures of the knee joint.

Output parameters related to knee joint behaviors, which are predicted by the knee model, may include but are not limited to knee joint kinematics, knee joint kinetics, and material stress. Specifically, tibiofemoral and patellofemoral positions and orientations, net joint loads, contact forces between articular surfaces or implant devices, and strain present in soft-tissue structures may be monitored during simulation. These parameters may be used individually or in combination, along with other non-model predicted parameters, to guide the optimized position and orientation for knee replacement devices.

Given a calibrated patient-specific knee model and implant position/orientation, a dynamic simulation may be performed to yield patient-specific knee kinematics and dynamics. Furthermore, the resulting knee kinematics and dynamics may be compared against a surgeon-defined list of objectives, such as symmetric medial/lateral contact forces, or a desired center of pressure, and report this information to a computer display. The surgeon may then manually adjust his/her implant plan and recalculate the implant planning score.

According to one embodiment, a nonlinear optimization method may be established to determine the optimal implant position to minimize a set of surgeon-defined objectives while satisfying a particular set of constraints. A weighted cost or score may be defined based on the aggregate sum of the implant positioning metrics. Implant positioning metrics may be divided into categories, such as mechanical alignment metrics (e_{M}), soft-tissue balancing metrics (e_{S}), and functional outcome metrics (e_{F}). Mechanical alignment metrics may include, but are not limited to, mechanical axis alignment, trans-epicondylar axis alignment, joint-line restoration, distance from a desired posterior slope, patella alta/baja, distance from a nominal Q angle, and/or minimizing the total bone resection. Soft-tissue balancing metrics may include, but are not limited to, balancing the medial/lateral ligament tension, balancing the medial/lateral flexion and/or extension gaps, and balancing the medial/lateral tibiofemoral and/or patellofemoral contact forces. Functional outcome metrics may include, but are not limited to, post-operative kinematic measures, such as the passive envelope of knee motion or knee laxity, knee flexion, femoral rollback, paradoxical motion, varus/valgus lift-off, and patella tracking, post-operative dynamic measures, such as medial/lateral center-of-pressure locations, and implant measures, such as bearing life expectancy. Optimization constraints may include satisfying the manufacturer's recommended implant alignment pose.

In an exemplary embodiment, the cost function may be written as the sum of cost functions for each implant pose category, such that

e=e_{M}+e_{S}+e_{F}

where e_{M}, e_{S}, and e_{F}, represent the cost functions comprising the mechanical alignment metrics, soft-tissue balancing metrics, and functional outcome metrics, respectively.

### Mechanical Alignment Metrics

The mechanical alignment metrics, e_{M}, may take many forms, including but not limited to a weighted sum of the following error functions: mechanical axis alignment, trans-epicondylar axis alignment, joint-line restoration, distance from a desired posterior slope, patella alta/baja, distance from a nominal Q angle, and/or minimizing the total bone resection.

Mechanical Axis Alignment:

e_{MA}=cos⁻¹(\vec{m}_{I}·\vec{m}_{B}),

where m_{I} represents the mechanical axis of the implant, and m_{B} represents the mechanical axis of the bone. This error is equivalent to the angle between these two axes.

Trans-Epicondylar Axis Alignment:

e_{TA}=cos⁻¹(\vec{t}_{I}·\vec{t}_{B}),

where t_{I} represents the trans-epicondylar axis of the implant, and t_{B} represents the trans-epicondylar axis of the bone.

Posterior-Slope Alignment:

e_{PS}=cos⁻¹(\vec{n}_{I}·\vec{n}_{D}),

where n_{I} represents the normal to the tibia baseplate, and n_{D} represents the normal to the plane defined by the desired posterior slope, often defined as a 3-5° rotation from the axial plane.

Joint-Line Preservation:

$${e}_{JL} = \frac{1}{N} {\sum }_{i = 1}^{N} {cos}^{- 1} ( {{x}^{->}}_{I} ( i ) \cdot {{x}^{->}}_{D} ( i ) ) ,$$

where x_{I}(i) represents the instantaneous axis of rotation of the implant for sample i, and x_{D}(i) represents the desired axis of rotation for sample i, defined from a priori data.

Patella Alta/Baja:

$${e}_{PA} = {\sum }_{i = 1}^{N} Φ ( i ) , \\ Φ ( i ) = \{ \begin{matrix}\vert p ( i ) - {p}_{MAX} \vert , & p ( i ) > {p}_{MAX} \\ 0 , & {p}_{MIN} < p ( i ) < {p}_{MAX} \\ \vert p ( i ) - {p}_{MIN} \vert , & p ( i ) < {p}_{MIN}\end{matrix}$$

where p represents the superior/inferior location of the patella with respect to the femoral coordinate system, and p_{MAX} and p_{MIN} are the maximum allowable superior and minimum allowable inferior positions of the patella.

Q-Angle:

e_{QA}=(Q_{I}−Q_{D})²,

where Q_{I} is the estimated Q-angle of the patella with the patient in a weight-bearing standing position following surgery, and Q_{D} is the desired Q-angle. The Q-angle cost function may also take the form of a piecewise polynomial function, defining an allowable range of Q-angles.

Resection Volume:

$${e}_{RV} = {\sum }_{i = 1}^{N} ( {v}_{I} ⋂ {v}_{B} ) .$$

where v_{I} and v_{B} are voxel representations of the implant and bone, respectively, and the operator ∩ determines the mathematical intersection of such voxel sets, defined by a minimum overlapping percentage.

A set of constant coefficients, α₁, α₂, . . . , α_{N}, may be used to scale or weight each of the respective cost function elements, in order to account for varying units and surgeon preferences, such that

e_{M}=α₁e_{MA}+α₂e_{TA}+ . . . +α_{N}e_{RV}.

### Soft-Tissue Balancing Metrics

The soft-tissue balancing metrics, e_{S}, may take many forms, including but not limited to a weighted sum of the following error functions: balancing the medial/lateral ligament tension, balancing the medial/lateral flexion and/or extension gaps, and balancing the medial/lateral tibiofemoral and/or patellofemoral contact forces.

Balancing MCL/LCL Ligament Tension:

$${e}_{ML} = \frac{1}{N} {\sum }_{i = 1}^{N} {( {f}_{MCL} ( i ) - {f}_{LCL} ( i ) )}^{2} ,$$

where f_{MCL}(i) and f_{LCL}(i) represent the tension in the medial collateral ligament (MCL) and lateral collateral ligament (LCL), respectively.

Balancing the Medial/Lateral Flexion and Extension Gaps:

$${e}_{GAP} = \frac{1}{N} {\sum }_{i = 1}^{N} {( {x}_{M} ( i ) - {x}_{L} ( i ) )}^{2} ,$$

x_{M}(i) and x_{L}(i) represent the gap (+) or overlap (−) in the medial and lateral compartments for sample i, respectively.

Balancing the Medial/Lateral Tibiofemoral Contact Forces:

$${e}_{CF} = \frac{1}{N} {\sum }_{i = 1}^{N} {( {f}_{M} ( i ) - {f}_{L} ( i ) )}^{2} ,$$

f_{M}(i) and f_{L}(i) represent the medial and lateral tibiofemoral contact forces for sample i, respectively.

Minimizing the Patellofemoral Contact Forces:

$${e}_{PF} = \frac{1}{N} {\sum }_{i = 1}^{N} {( {f}_{PF} ( i ) )}^{2} ,$$

f_{PF}(i) represents the magnitude of the patellofemoral contact forces for sample i.

A set of constant coefficients, β₁, β₂, . . . β_{N}, may be used to scale or weight each of the respective cost function elements, in order to account for varying units and surgeon preferences, such that

e_{S}=β₁e_{ML}+β₂e_{CF}+ . . . +β_{N}e_{PF}.

### Functional Outcome Metrics

The functional outcome metrics, e_{F}, may take many forms, including but not limited to a weighted sum of the following error functions: post-operative kinematic measures, such as knee laxity, i.e. the passive envelope of knee motion, knee flexion, femoral rollback, paradoxical motion, varus/valgus lift-off, and patella tracking, post-operative dynamic measures, such as medial/lateral center-of-pressure locations, and implant measures, such as bearing life expectancy.

Knee Laxity:

$${e}_{KL} = {( \frac{{a}_{D} - {a}_{I}}{{a}_{D}} )}^{2}$$

where a_{I} represents the anterior tibial translation (ATT), for example during a Lachman's knee laxity examination, and a_{D} represents the desired knee laxity. In an alternative formulation, the cost function may be expressed as a piecewise polynomial, such that the resulting cost is zero for an allowable range of anterior tibial translations.

Knee Flexion:

$${e}_{KF} = {( \frac{{θ}_{D} - {θ}_{I}}{{θ}_{D}} )}^{2}$$

where θ_{I} represents the maximum achievable flexion angle in degrees, and θ_{D} represents the maximum desired flexion angle, which may for example be 150 degrees.

Femoral Rollback:

$${e}_{FR} = {( \frac{{y}_{D} - {y}_{I}}{{y}_{D}} )}^{2}$$

where y_{I} represents the femoral rollback, defined as the posterior translation of the femur in the plane of the tibial baseplate, and y_{D} represents the desired femoral rollback.

Paradoxical Motion:

e_{FR}=((φ_{MAX})²

where φ_{MAX} represents the maximum angle through which the femur rotates about the lateral compartment, considered a paradoxical motion to the natural medial rotation of the knee joint.

Varus/Valgus Lift-Off:

$${e}_{vv} = \frac{1}{N} {\sum }_{i = 1}^{N} ( {{x}_{VR} ( i )}^{2} + {{x}_{VG} ( i )}^{2} ) ,$$

where x_{VG}(i) and x_{VG}(i) represent the varus and valgus liftoff in the lateral and medial compartments, respectively, for sample i.

Patella Tracking:

e_{PT}=max ∥\vec{α}_{PT}(i)∥

where e_{PT} represents the maximum patella acceleration for all samples during a pre-determined patient activity, such as gait.

Medial/Lateral Center-of-Pressure Locations:

$${e}_{CP} = \frac{1}{N} {\sum }_{i = 1}^{N} ( ‖ {{p}^{->}}_{MP} ( i ) - {{p}^{->}}_{MP , D} ( i ) ‖ + ‖ {{p}^{->}}_{LP} ( i ) - {{p}^{->}}_{LP , D} ( i ) ‖ ) ,$$

where p_{MP}(i) and p_{LP}(i) represent the estimated medial and lateral center-of-pressure (COP) locations for sample i, p_{MP,D}(i) and p_{LP,D}(i) represent the desired medial and lateral COP locations for sample i, and N is the number of samples for a particular activity.

Bearing Life Expectancy:

$${e}_{BL} = \{ \begin{matrix}\frac{{L}_{D} - L}{{L}_{D}} , & L < {L}_{D} \\ 0 , & L > {L}_{D}\end{matrix}$$

where L_{D} represents the desired bearing life expectancy, e.g. 15 years, and L represents the expected bearing life expectancy based on a dynamic simulation.

A set of constant coefficients, γ₁, γ₂, . . . γ_{N}, may be used to scale or weight each of the respective cost function elements, in order to account for varying units and surgeon preferences, such that

e_{F}=γ₁e_{ML}+γ₂e_{AP}+ . . . +γ_{N}e_{PF}.

The optimal implant pose may be determined by solving for the argument of the minimum of the preceding cost function through a global optimization method, such that

\vec{x}=arg min_{\vec{x}}e(\vec{x}),

subject to

hᵢ(x)=0

gⱼ(x)≦0,

where x represent the 6 DoF position and orientation of the implant with respect to the local bone coordinate system, h(x) represents a set of equality constraints, and g(x) represents a set of inequality constraints. The inequality constraints may be framed such that they incorporate the manufacturer's recommended range of implant placement. In the preferred embodiment, the global optimization method may be a genetic algorithm to avoid local minima, such that future offspring are computed through both randomly selected crossovers and mutations of the parent population.

According to an exemplary embodiment, the present invention calculates the sensitivity of the final solution to implant positioning errors. Implant positioning errors for computer-assisted and robot-assisted surgical system may vary from 2-3 mm, and 2-3 degrees. Therefore, it is beneficial to evaluate the implant poses in the area surrounding the final target solution, and confirm that they are also acceptable.

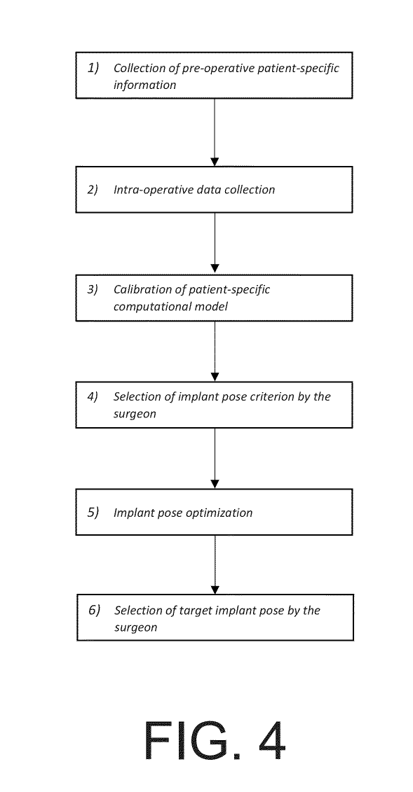

FIG. 4 illustrates an exemplary process for using the simulation system in accordance with the disclosed embodiments. As illustrated in FIG. 4, the process may include the steps of: 1) collecting pre-operative patient-specific information; 2) collecting intra-operative data; 3) calibrating a patient-specific computational model based on at least one of the pre-operative patient-specific data and intra-operative data; 4) selecting implant planning criterion; 5) optimizing the implant pose based, at least in part, on the implant planning criteria, patient-specific information, and intra-operative data; and 6) selecting/validating target implant pose by the surgeon.

In the first step, a CT scan or MRI may be performed to ascertain the patient-specific bony and soft tissue geometry of the patient. A conventional segmentation and 3D reconstruction technique may be applied to determine a 3D bone model. The soft tissue geometry, such as ligament origin and insertion sites, may be manually selected from the series of MRI slices.

In the second step, patient-specific intra-operative data may be collected from one or more sensing devices, such as an external tracking system, inter-body force sensing device, or external reaction force sensing device.

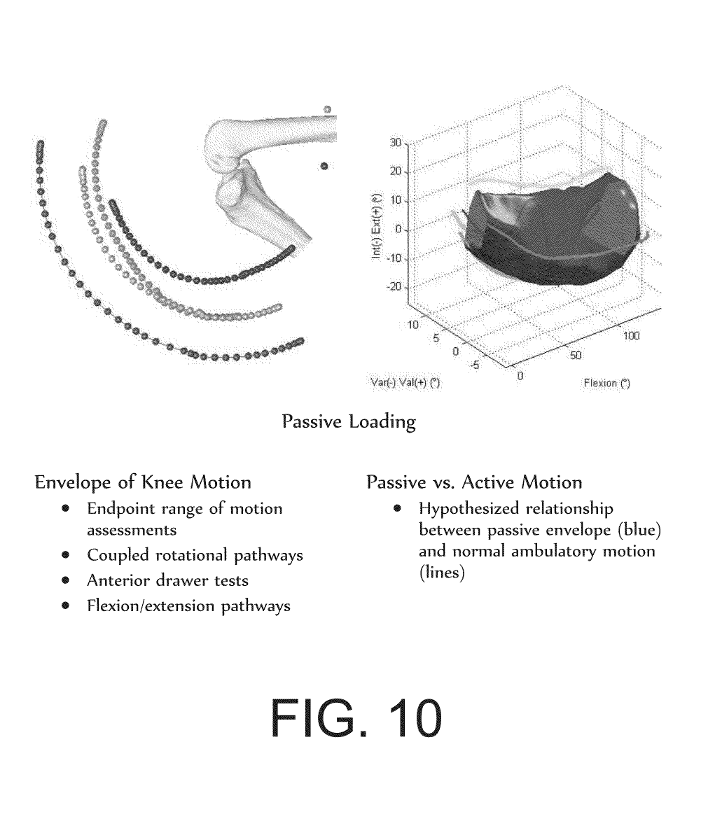

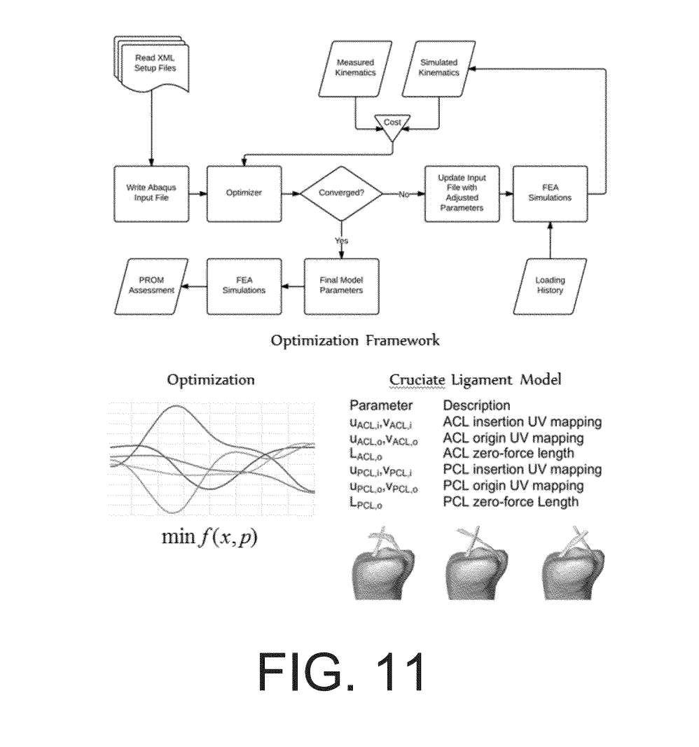

In the third step, a patient-specific computational model is determined from measured data and pre-operative patient-specific information, such as 3D models. Data may be collected intra-operatively from passive manipulation of the knee as shown in FIG. 10. The calibration may be achieved through a constrained non-linear optimization, where the cost function may include the displacement errors from a forward dynamics simulation, or the force/torque errors from an inverse dynamic simulation. The design inputs to the optimization are the patient-specific model parameters, such as material properties or nominal ligament lengths as illustrated in FIG. 11.

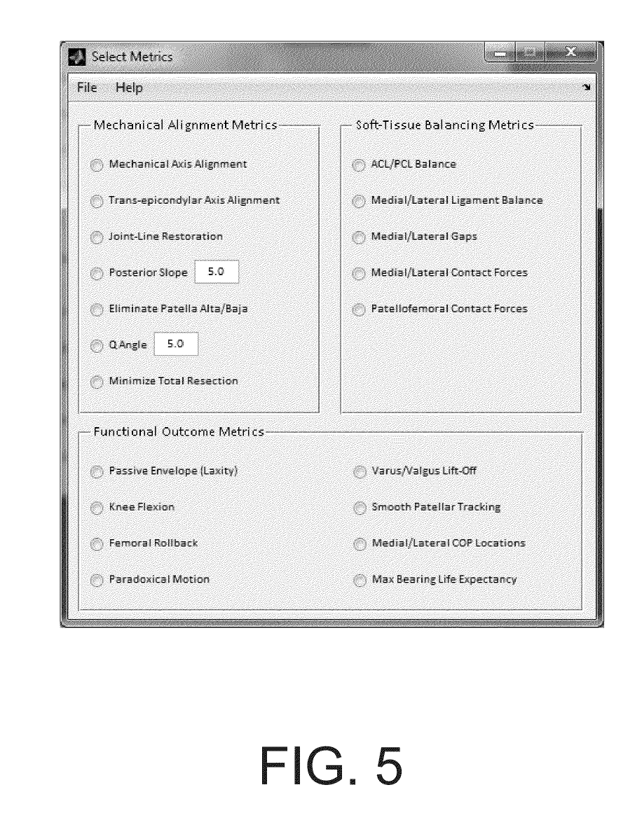

In the fourth step, the surgeon may select one or more criteria for determining the implant pose, such as mechanical alignment metrics, soft-tissue balancing metrics, and/or functional outcome metrics (see preceding sections). A screen shot of an exemplary graphical user interface (GUI) associated with software that allows the surgeon to select one or more criteria for determining the implant pose is illustrated in FIG. 5.

In the fifth step, an optimization routine determines the optimal implant pose given the combination of metrics and constraints selected by the surgeon. A monitor may then display a 3D model of the patient's anatomy and the resultant implant pose.

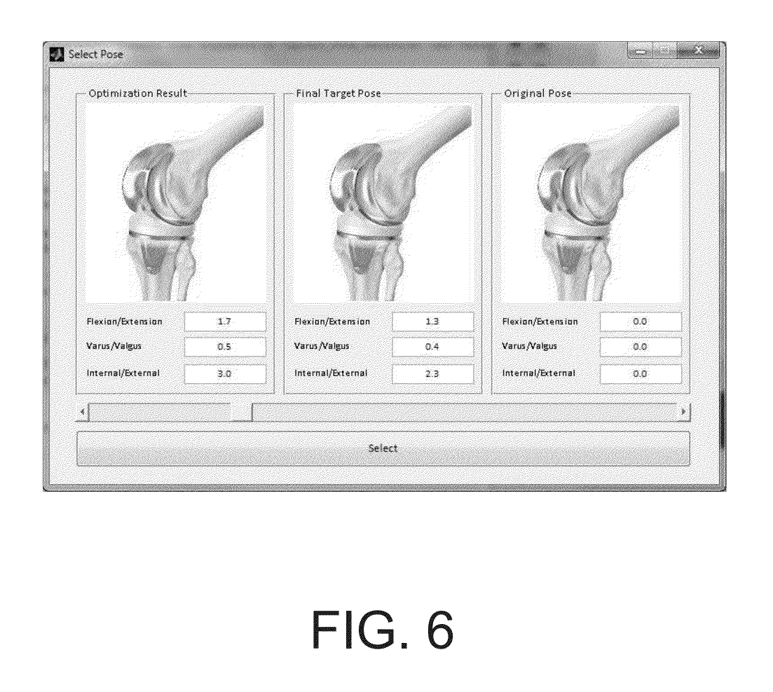

In a sixth step, the surgeon may adjust the implant position based on his experience or confirm the optimization result. A screen shot of an exemplary graphical user interface (GUI) associated with software that allows the surgeon to adjust the implant position is shown in FIG. 6. This page may display both the original and optimized implant plan, and enable the surgeon to selectively tune the target pose based on some combination of these 2 solutions (i.e. a linearly weighted combination). A computational simulation may be performed based on this adjusted target pose to compute and display the implant positioning metrics, such as mechanical axis alignment, medial/lateral contact forces, and knee range of motion for the final target pose.

In an alternative embodiment, the aforementioned technique may be used in other types of orthopaedic surgery, such as total hip arthroplasty (THA), total shoulder arthroplasty (TSA), total disc replacement (TDR), and other joint replacement surgeries, which may benefit from an intelligent implant planning strategy considering both kinematic and kinetic measures. Furthermore, this technique may also be applied to plan the resection region in surgeries not requiring a permanent implant, such as a femoroacetabular impingement (FAI) surgery, laminectomy, or subacromial impingement surgery. For example, in an FAI surgery, a patient-specific computational model of the hip may be calibrated from pre- and intra-operative data, such as bone geometries, kinematics, and external reaction forces. A surgeon may select one or more criteria for determining the femoral and/or acetabular resection, such as maximizing range of motion, reducing bone loss, or minimizing bone stress. An optimization routine may then compute an optimal resection plan to eliminate cam and/or pincer impingement based on the patient-specific computational model and combination of metrics and constraints selected by the surgeon.

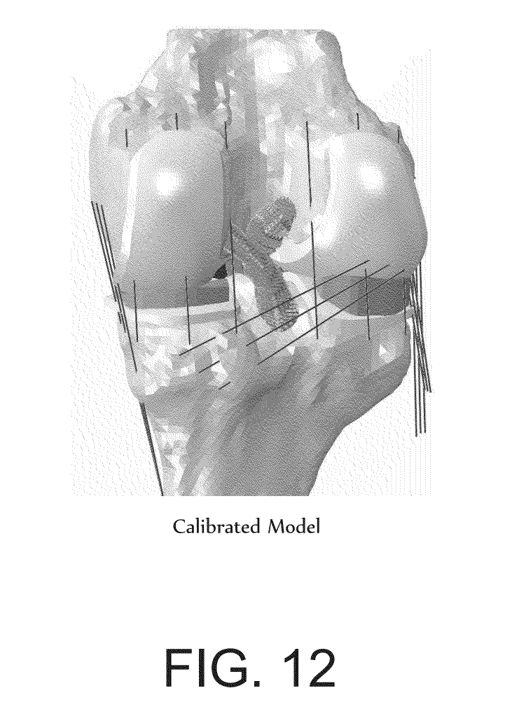

FIG. 12 illustrates an exemplary processor-based computer system, on which certain methods and processes consistent with the disclosed force sensor-based may be implemented. Computer 120, as schematically illustrated in FIG. 12, may include one or more hardware and/or software components configured to collect, monitor, store, analyze, evaluate, distribute, report, process, record, and/or sort information associated with a computer-assisted surgical system shown and illustrated in the disclosed embodiments. For example, computer 120 may be programmed to perform the simulations, optimizations, and analyses, as described in certain disclosed embodiments.

According to an exemplary embodiment, controller 120 may include one or more hardware components such as, for example, a central processing unit (CPU) 121, a random access memory (RAM) module 122, a read-only memory (ROM) module 123, a storage 124, a database 125, one or more input/output (I/O) devices 126, and an interface 127. Alternatively and/or additionally, controller 120 may include one or more software components such as, for example, a computer-readable medium including computer-executable instructions for performing a method associated with collision warning system 111. It is contemplated that one or more of the hardware components listed above may be implemented using software. For example, storage 124 may include a software partition associated with one or more other hardware components of controller 120. Controller 120 may include additional, fewer, and/or different components than those listed above. It is understood that the components listed above are exemplary only and not intended to be limiting.

CPU 121 may include one or more processors, each configured to execute instructions and process data to perform one or more functions associated with controller 120. As illustrated in FIG. 1, CPU 121 may be communicatively coupled to RAM 122, ROM 123, storage 124, database 125, I/O devices 126, and interface 127. CPU 121 may be configured to execute sequences of computer program instructions to perform various processes, which will be described in detail below. The computer program instructions may be loaded into RAM 122 for execution by CPU 121.

RAM 122 and ROM 123 may each include one or more devices for storing information associated with an operation of controller 120 and/or CPU 121. For example, ROM 123 may include a memory device configured to access and store information associated with controller 120, including information for identifying, initializing, and monitoring the operation of one or more components and subsystems of controller 120. RAM 122 may include a memory device for storing data associated with one or more operations of CPU 121. For example, ROM 123 may load instructions into RAM 122 for execution by CPU 121.

Storage 124 may include any type of mass storage device configured to store information that CPU 121 may need to perform processes consistent with the disclosed embodiments. For example, storage 124 may include one or more magnetic and/or optical disk devices, such as hard drives, CD-ROMs, DVD-ROMs, or any other type of mass media device.

Database 125 may include one or more software and/or hardware components that cooperate to store, organize, sort, filter, and/or arrange data used by controller 120 and/or CPU 121. For example, database 125 may store predetermined operator reaction time information associated with different conditions (e.g., fog, rain, snow, time-of-day, etc.) at different speeds. CPU 121 may access the information stored in database 125 to determine a threshold warning distance for collision warning system 111. It is contemplated that database 125 may store additional and/or different information than that listed above.

I/O devices 126 may include one or more components configured to communicate information with a user associated with controller 120. For example, I/O devices may include a console with an integrated keyboard and mouse to allow a user to input parameters associated with controller 120. I/O devices 126 may also include a display including a graphical user interface (GUI) for outputting information on a monitor. I/O devices 126 may also include peripheral devices such as, for example, a printer for printing information associated with controller 120, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 127 may include one or more components configured to transmit and receive data via a communication network, such as the Internet, a local area network, a workstation peer-to-peer network, a direct link network, a wireless network, or any other suitable communication platform. For example, interface 127 may include one or more modulators, demodulators, multiplexers, demultiplexers, network communication devices, wireless devices, antennas, modems, and any other type of device configured to enable data communication via a communication network.

It will be apparent to those skilled in the art that various modifications and variations can be made to the disclosed systems and associated methods for determining a change in a parameter associated with a joint caused by a modification of a portion of the joint. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

What is claimed is:

1. A method for determining implant position and orientation, the method comprising:
recording at least one kinematic or kinetic parameter during a passive loading of a portion of an anatomy associated with the joint of a patient;
calibrating a patient-specific software model associated with a patient's joint based, at least in part, on the at least one kinematic or kinetic parameter;
receiving, by the processor, one or more user-selected parameters associated with joint performance; and
simulating performance of the patient's joint using the calibrated patient-specific model and the one or more user-selected parameters; and
providing, by the processor, information indicative of at least one of a recommended implant position or a recommended implant orientation, based on the simulated performance.

2. The method of claim 1, wherein calibrating the patient specific software model is further based, at least in part, on one or more of: a geometry of the patient's joint, a kinematic parameter of the patient's joint, or an external reaction forces associated with the patient's joint.

3. The method of claim 2, wherein at least one of the one or more of the geometry, the kinematic parameter, and the external reaction forces is determined intra-operatively during a joint replacement procedure.

4. The method of claim 2, wherein simulating performance of the patient's joint includes performing a non-linear optimization using the patient specific model and received user-selected performance parameters.

5. The method of claim 4, wherein the non-linear optimization is based on cost functions associated with a mechanical alignment metric, a soft-tissue balancing metric, or a functional outcome metric.

6. The method of claim 5, wherein a mechanical alignment metric includes at least one of a mechanical axis alignment, a trans-epicondylar axis alignment, a posterior-slope alignment, a joint-line preservation parameter, a patella alto/baja parameter, a Q-angle, or a resection volume.

7. The method of claim 5, wherein the soft-tissue balancing metric includes at least one of an MCL/LCL ligament tension parameter, a medial/lateral tibiofemoral contact force parameter, a medial/lateral flexion and extension gap parameter, and a patellofemoral contact force parameter.

8. The method of claim 5, wherein the functional outcome metric includes at least one of a knee laxity parameter, a knee flexion parameter, a femoral rollback parameter, a paradoxical motion parameter, a varus/valgus lift-off parameter, a patella tracking parameter, a medial/lateral center-of-pressure location, and a bearing life expectancy parameter.

9. A method for devising a resection plan for reducing joint impingement, comprising:
calibrating a patient-specific software model associated with a patient's joint based, at least in part, on one or more of: a geometry of the patient's joint, a kinematic parameter of the patient's joint, or an external reaction forces associated with the patient's joint;
receiving, by the processor, one or more user-selected parameters associated with joint performance; and
simulating performance of the patient's joint using the calibrated patient-specific model and the one or more user-selected parameters; and
generating information indicative of a resection plan associated with the patient's joint, based on the simulated performance.

10. The method of claim 9, wherein the joint impingement may be at least one of a femoroacetabular impingement, neural impingement, or subacromial impingement.

11. The method of claim 9, wherein at least one of the one or more of the geometry, the kinematic parameter, and the external reaction forces is determined intra-operatively during a surgical procedure.

12. The method of claim 9, wherein the one or more user-selected parameters includes information indicative of a desire to increase range of motion associated with a post-operative joint, information indicative of a desire to minimize bone loss due to the resection, and/or information indicative of a desire to limit bone stress due to the resection.

13. The method of claim 9, wherein simulating performance of the patient's joint includes performing a non-linear optimization using the patient specific model and received user-selected performance parameters.

14. The method of claim 13, wherein the non-linear optimization is based on cost functions associated with a range of motion metric, a bone loss metric, or a bone stress metric.

15. An apparatus for measuring external reaction forces used in calibrating a patient-specific model, comprising:
a leg holding device configured to receive at least a portion of a patient's lower leg;
a plurality of sensors coupled to the leg holding device and configured to measure an external force applied to the patient's lower leg; and
a tracking device coupled to the leg holder and configured to locate at least one of a position or an orientation of the leg holding device relative to an anatomical feature of the patient.

16. The apparatus of claim 15, wherein the leg holding device includes a rigid boot for receiving therein at least a portion of the patient's lower leg, wherein the rigid boot includes a plurality of handles coupled to a body portion of the rigid boot, the plurality of handles for manipulating a position of the patient's lower leg, and wherein at least a first sensor of the plurality of sensors is coupled to a first one of the handles and at least a second of the plurality of sensors is coupled to a second one of the handles, the first and second sensors configured to measure a force applied to the first and second handle, respectively.

17. The apparatus of claim 15, wherein the leg holding devices includes a robotic manipulator for actively manipulating a position of the patient's lower leg and for measuring the applied forces.

18. The apparatus of claim 15, wherein each of the plurality of sensors includes a strain gauge configured to measure a force or torque applied to the leg-holding device in at least 6 degrees-of-freedom.

19. The apparatus of claim 15, further comprising a wireless communication device in data communication with an off-board controller and configured to transmit external force information collected from the plurality of sensors and position or orientation information collected from the tracking device to the off-board controller.
